(19) United States
(12) Patent Application Publication
(10) Pub. No.: US 2007/0099958 A1
(43) Pub. Date: May 3, 2007

(54) Protease inhibitors

(75) Inventors: Jon Bondebjerg, Copenhagen O (DK); Henrik Fuglsang, Lyngby (DK); Lars Naerum, Hellerup (DK)

Correspondence Address: BIRCH STEWART KOLASCH & BIRCH, PO BOX 747, FALLS CHURCH, VA, 22040-0747, US

(73) Assignee: ProZymex A/S, Horsholm (DK)

(21) Appl. No.: 10/560,924

(22) PCT Filed: Jun. 17, 2004
(86) PCT No.: PCT/DK04/00421

§ 371 Date: Dec. 13, 2006

Related U.S. Application Data
(60) Provisional application No. 60/479,961, filed on Jun. 19, 2003.

(30) Foreign Application Priority Data
Jun. 18, 2003 (DK) PA 2003 00905

(51) Int. Cl. A61K 31/445; A61K 31/40; A61K 31/277; C07C 255/60

(52) U.S. Cl. 514/317; 514/531; 558/410; 514/408; 548/561; 546/229

(57) ABSTRACT

A peptidyl nitrile of the general formula (I) or a pharmaceutically acceptable salt or prodrug thereof, is capable of selectively inhibiting dipeptidyl-peptidase (DPP-I), also known as cathepsin C. A compound of the invention is useful as an active substance for the treatment of inflammation, type 2 diabetes, asthma, severe influenza, respiratory syncytial virus infection, CD8 T cell inhibition, inflammatory bowel diseases, psoriasis, atopic dermatitis, Papillon Lefevre syndrome, Haim Munk syndrome, gun disease, periodontitis, rheumatoid arthritis, Huntington's disease, Chagas' disease, Alzheimer's disease, sepsis or for application in target cell apoptosis.

### FIELD OF THE INVENTION

The present invention relates to novel protease inhibitors, more specifically to inhibitors of cysteine and/or serine proteases useful in the treatment/prevention of inflammation, diabetes and similar diseases in which proteases are involved, especially mast cell inflammatory mediated diseases. More specifically the invention relates to peptidyl nitriles capable of selectively inhibiting dipeptidyl-peptidase I (DPPI), also known as cathepsin C, an enzyme that cleaves a dipeptide from the N terminus of a polypeptide chain.

### BACKGROUND OF THE INVENTION

Dipeptidyl peptidase-I (DPP-I; EC 3.4.14.1) also known as cathepsin C is a lysosomal cysteine protease belonging to the papain family. The enzyme is constitutively expressed in many tissues with highest levels in lung, kidney, liver and spleen. The cDNAs encoding rat, human and murine DPP-I have been cloned and sequenced and showed that the enzyme is highly conserved. DPP-I is synthesized as an inactive precursor (Zymogen), and is activated by a non-autocatalytic excision of an internal activation peptide within the N-terminal propeptide. Once activated, DPP-I catalyzes the removal of dipeptides from the N-terminal end of polypeptide substrates with broad specificity. The pH optimum lies in the region of 5-7 using human DPP-1. Furthermore, DPP-I is oligomeric with little amino acid sequence homology compared to the exopeptidases cathepsin B, H, L, O and S which in addition are monomeric. Recent data suggests that, beside of being an important enzyme in lysosomal protein degradation, DPP-I also functions as a key enzyme in the activation of granule serine peptidases in cytotoxic T lymphocytes and natural killer cells (granzymes A and B), mast cells (chymase and tryptase), and neutrophils (cathepsin G and elastase).

Mast cells are found in many tissues, but are present in greater numbers along the epithelial linings of the body, such as the skin, respiratory tract and gastrointestinal tract. Mast cells are also located in the perivascular tissue surrounding small blood vessels. In humans, two types of mast cells have been identified. The T-type, which expresses only tryptase, and the MC-type, which expresses both tryptase and chymase. In humans, the T-type mast cells are located primarily in alveolar tissue and intestinal mucose while the TC-type cells predominate in skin and conjuctiva. Mast cells can release a range of potent inflammatory mediators including cytokines, leukotrienes, prostaglandins, histamine and proteoglycans, but among the most abundant products of mast cell activation are the serine proteases of the chymotrypsin family; tryptase and chymase. These proteases are situated in the mast cell lysosomes as fully active enzymes. The exact site of tryptase and chymase activation from zymogen precursors is not known, but the Golgi apparatus might play a role in that regard. DPP-I, which is particular abundant in mast cells, seems to be the key enzyme responsible for activation of chymase and tryptase. Moreover, tryptase and chymase are emerging as important mediators of allergic diseases. After secretion from activated mast cells, there is evidence that these proteases are heavily involved in processes of inflammation, tissue remodelling, bronchoconstriction and mucus secretion, which have made these proteases attractive for therapeutic intervention. Mast cells seem also to play a role in angiogenesis since these cells accumulate in many angiogenesis-dependent situations. Moreover, several mast cell mediators (e.g. histamine, chymase, VEGF and bFGF) are found to be angiogenic and regulate endothelial cell proliferation and function.

Neutrophils cause considerable damage in a number of pathological conditions. When activated, neutrophils secrete destructive granular enzymes including elastase and cathepsin G and undergo oxidative bursts to release reactive oxygen intermediates. Numerous studies have been conducted on each of these activating agents in isolation. Pulmonary emphysema, cystic fibrosis and rheumatoid arthritis are just some examples of pathological conditions associated with the potent enzymes elastase and cathepsin G.

The strong evidence associating tryptase and chymase with a number of mast cell mediated allergic, immunological and inflammatory diseases, and the fact that cathepsin G and elastase also seem to play important roles in these types of diseases points out DPP-I as an attractive target enzyme for therapeutic intervention against the above mentioned diseases, due to its central role in activating these proteases.

WO 9924460 to Novartis AG discloses dipeptide nitrile inhibitors of cysteine cathepsins.

WO 0187828 A1 to Novartis AG discloses N-substituted peptidyl nitriles as cysteine cathepsin inhibitors.

WO 0055126 to Axys Pharmaceuticals discloses N-cyanomethyl amides, which are cysteine protease inhibitors.

U.S. Pat. No. 5,602,102 to The University of Texas discloses inhibitors of dipeptidyl peptidase 1.

WO03/022871A2 to Probiodrug discloses inhibitors of dipeptidyl peptidase 1.

### SUMMARY OF THE INVENTION

The present invention relates to compounds of the general formula (I) 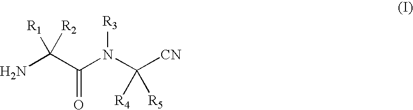
wherein R₁, R₂, R₃, R₄, and R₅ are as defined in the detailed part of this description.

It is contemplated that the compounds of the invention are useful for the treatment of inflammation or type2 diabetes, particularly for treatment of prevention of mast cell inflammatory mediated diseases such as asthma, severe influenza, respiratory syncytial virus infection, CD8 T cell inhibition, inflammatory bowel diseases, psoriasis, atopic dermatitis, Papillon Lefevre syndrome, Haim Munk syndrome, gum disease, periodontitis, rheumatoid arthritis, Huntington's disease, Chagas' disease, Alzheimer's disease and sepsis. The compounds of the present invention are especially applicable in target cell apoptosis.

In accordance with the present invention there is provided compounds, which are contemplated as being useful as in vitro and in vivo diagnostic tools.

It is an object of the invention to provide novel compounds having pharmacological activity as inhibitors of proteases such as serine and/or cysteine proteases. In a specific embodiment, the compounds of the invention are cysteine protease inhibitors, particularly selective cysteine protease inhibitors. More specifically, the compounds of the invention are inhibitors of cysteine proteases of the papain superfamily such as dipeptidyl-peptidase I.

Further objects will become apparent from the following description.

### DETAILED DESCRIPTION OF THE INVENTION

Definitions

The term “DPP-I” or “DPPI” as used herein is intended to mean dipeptidyl-peptidase I (EC 3.4.14.1) also known as cathepsin C, cathepsin J, dipeptidyl aminopeptidase I and dipeptidyl transferase. DPPI cleaves a dipeptide Xaa-Xbb from the N terminus of a polypeptide chain Xaa-Xbb-Xcc-[Xxx]ₙ, except when Xaa is Arg or Lys, or when Xbb or Xcc is Pro.

The term “treatment” is defined as the management and care of a patient for the purpose of combating the disease, condition, or disorder and includes the administration of a compound of the present invention to prevent the onset of the symptoms or the complications, or alleviating the symptoms or the complications, or eliminating the disease, condition, or disorder.

As used herein, alone or in combination, the term “C₁₋₆ alkyl” denotes a straight or branched, saturated hydrocarbon chain having from one to six carbon atoms. C₁₋₆ alkyl groups include, but are not limited to, methyl, ethyl, n-propyl, iso-propyl, n-butyl, iso-butyl, sec-butyl, tert-butyl, n-pentyl, iso-pentyl, 2-methylbutyl, 3-methylbutyl, n-hexyl, iso-hexyl, 4-methylpentyl, neopentyl, 2,2-dimethylpropyl and the like.

As used herein, alone or in combination, the term “C₂6 alkenyl” denotes a straight or branched, unsaturated hydrocarbon chain having from two to six carbon atoms and at least one double bond. C₂6 alkenyl groups include, but are not limited to, vinyl, 1-propenyl, allyl, iso-propenyl, n-butenyl, n-pentenyl, n-hexenyl and the like.

As used herein, alone or in combination, the term “C₂6 alkynyl” denotes a straight or branched, unsaturated hydrocarbon chain having from two to six carbon atoms and at least one triple bond. C₂₋₄ alkynyl groups include, but are not limited to, —C≡CH, —C≡CCH₃, —CH₂C≡H, —CH₂—CH₂C≡H, —CH(CH₃)C≡CH and the like.

The term “C₁₋₆ alkoxy” in the present context designates a group O—C₁₋₆ alkyl used alone or in combination, wherein C₁₋₆ alkyl is as defined above. Examples of straight alkoxy groups are methoxy, ethoxy, propoxy, butoxy, pentoxy and hexoxy. Examples of branched alkoxy are iso-propoxy, sec-butoxy, tert-butoxy, iso-pentoxy and iso-hexoxy.

Examples of cyclic alkoxy are cyclopropyloxy, cyclobutyloxy, cyclopentyloxy and cyclohexyloxy.

The term “C₁₋₆ alkylthio” in the present context designates a group —S—C₁₋₆ alkyl wherein C₁₋₄ alkyl is as defined above. Representative examples include, but are not limited to, methylthio, ethylthio, n-propylthio, isopropylthio, butylthio, isobutylthio, sec-butylthio, tert-butylthio, n-pentylthio, isopentylthio, neopentylthio, tert-pentylthio, n-hexylthio, isohexylthio and the like.

The term “C₁₋₆ alkylcarbonyl” in the present context designates a group —(CO)—C₁₋₆ alkyl wherein C₁₋₄ alkyl is as defined above. Representative examples include, but are not limited to, methylcarbonyl, ethylcarbonyl, n-propylcarbonyl, isopropylcarbonyl, butylcarbonyl, isobutylcarbonyl, sec-butylcarbonyl, tert-butylcarbonyl, n-pentylcarbonyl, isopentylcarbonyl, neopentylcarbonyl, tert-pentylcarbonyl, n-hexylcarbonyl, isohexylcarbonyl and the like.

The term “C₁₋₆ alkylsulfonyl” in the present context designates a group —(SO)₂—C₁₋₆-alkyl wherein C₁₋₆-alkyl is as defined above. Representative examples include, but are not limited to, methylsulfonyl, ethylsulfonyl, n-propylsulfonyl, isopropylsulfonyl, butylsulfonyl, isobutylsulfonyl, sec-butylsulfonyl, tert-butylsulfonyl, n-pentylsulfonyl, isopentylsulfonyl, neopentylsulfonyl, tert-pentylsulfonyl, n-hexylsulfonyl, isohexylsulfonyl and the like.

The term “C₁₋₆ N-alkylamide” in the present context designates a group —(CO)NH—C₁₋₆alkyl, wherein C₁₋₆ alkyl is as defined above. Representative examples include, but are not limited to, N-methylamide, N-ethylamide, N-propylamide, N-butylamide, N-pentylamide and N-hexylamide.

The term “dialkylamino C₁₋₆ alkyl” as used herein designates a group di-C₁₋₄alkyl-N—C₁₋₆alkyl, wherein C₁₋₆ alkyl is as defined above. Representative examples include, but are not limited to, dimethylaminomethyl.

The term “C₃₋₁₀ cycloalkyl” as used herein denotes a radical of one or more saturated mono-, bi-, tri- or spirocyclic hydrocarbon having from three to ten carbon atoms. Examples include, but are not limited to, cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₁₀-cycloalkylcarbonyl” as used herein represents a group —(CO)—C₃₋₁₀-cycloalkyl wherein C₃₋₁₀-cycloalkyl is as defined above.

The term “C₅₋₁₀ cycloalkenyl” as used herein denotes a radical of one or more saturated cyclic hydrocarbon having from five to ten carbon atoms and at least one double bond. Examples include, but are not limited to, cyclopentenyl and cyclohexenyl and the like.

The term “C₃₋₇ heterocycloalkyl” as used herein denotes a radical of a totally saturated heterocycle like a cyclic hydrocarbon containing one or more heteroatoms selected from nitrogen, oxygen and sulphur independently in the cycle. Examples of heterocycles include, but are not limited to, pyrrolidine (1-pyrrolidine, 2-pyrrolidine, 3-pyrrolidine, 4-pyrrolidine, 5-pyrrolidine), pyrazolidine (1-pyrazolidine, 2-pyrazolidine, 3-pyrazolidine, 4-pyrazolidine, 5-pyrazolidine), imidazolidine (1-imidazolidine, 2-imidazolidine, 3-imidazolidine, 4-imidazolidine, 5-imidazolidine), thiazolidine (2-thiazolidine, 3-thiazolidine, 4-thiazolidine, 5-thiazolidine), piperidine (1-piperidine, 2-piperidine, 3-piperidine, 4-piperidine, 5-piperidine, 6-piperidine), piperazine (1-piperazine, 2-piperazine, 3-piperazine, 4-piperazine, 5-piperazine, 6-piperazine), morpholine (2-morpholine, 3-morpholine, 4-morpholine, 5-morpholine, 6-morpholine), thiomorpholine (2-thiomorpholine, 3-thiomorpholine, 4-thiomorpholine, 5-thiomorpholine, 6-thiomorpholine), 1,2-oxathiolane (3-(1,2-oxathiolane), 4-(1,2-oxathiolane), 5-(1,2-oxathiolane)), 1,3-dioxolane (2-(1,3-dioxolane), 3-(1,3-dioxolane), 4-(1,3-dioxolane)), tetrahydropyrane (2-tetrahydropyrane, 3-tetrahydropyrane, 4-tetrahydropyrane, 5-tetrahydropyrane, 6-tetrahydropyrane), hexahydropyradizine, (1-(hexahydropyradizine), 2-(hexahydropyradizine), 3-(hexahydropyradizine), 4-(hexahydropyradizine), 5-(hexahydropyradizine), 6-(hexahydropyradizine)).

The term “aryl” as used herein is intended to include carbocyclic aromatic ring systems. Aryl is also intended to include the partially hydrogenated derivatives of the carbocyclic systems enumerated below.

The term “heteroaryl” as used herein includes heterocyclic aromatic ring systems containing one or more heteroatoms selected from nitrogen, oxygen and sulphur such as furyl, thienyl, pyrrolyl, and is also intended to include the partially hydrogenated derivatives of the heterocyclic systems enumerated below.

Examples of “aryl” and “heteroaryl” includes, but are not limited to, phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), phenanthrenyl, fluorenyl, pentalenyl, azulenyl, biphenylenyl, thiophenyl (1-thienyl, 2-thienyl), furyl (1-furyl, 2-furyl), furanyl, thiophenyl, isoxazolyl, isothiazolyl, 1,2,3-triazolyl, 1,2,4-triazolyl, pyranyl, pyridazinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indolyl, oxadiazolyl, isoxazolyl, quinazolinyl, fluorenyl, xanthenyl, isoindanyl, benzhydryl, acridinyl, benzisoxazolyl, purinyl, quinazolinyl, quinolizinyl, quinolinyl, isoquinolinyl, quinoxalinyl, naphthyridinyl, phteridinyl, azepinyl, diazepinyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl, 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), isoquinolyl (1-isoquinolyl, 3-isoquinolyl, 4-isoquinolyl, 5-isoquinolyl, 6-isoquinolyl, 7-isoquinolyl, 8-isoquinolyl), quinolyl (2-quinolyl, 3-quinolyl, 4-quinolyl, 5-quinolyl, 6-quinolyl, 7-quinolyl, 8-quinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl)), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl)), indolyl (1-indolyl, 2-indolyl, 3-indolyl, 4-indolyl, 5-indolyl, 6-indolyl, 7-indolyl), indazolyl (1-indazolyl, 2-indazolyl, 3-indazolyl, 4-indazolyl, 5-indazolyl, 6-indazolyl, 7-indazolyl), benzimidazolyl, (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl). Non-limiting examples of partially hydrogenated derivatives are 1,2,3,4-tetrahydronaphthyl, 1,4-dihydronaphthyl, pyrrolinyl, pyrazolinyl, indolinyl, oxazolidinyl, oxazolinyl, oxazepinyl and the like.

The term “C₁₋₆-alkylaryl” as used herein refers to an aryl group as defined above attached through a C₁₋₆-alkyl group as defined above having one, two, three, four, five or six carbon atoms.

The term “C₁₋₆-alkylheteroaryl” as used herein refers to a heteroaryl group as defined above attached through a C₁₋₆alkyl group as defined above having one, two, three, four, five or six carbon atoms.

The term “aroyl” as used herein represents a group —(CO)-aryl wherein aryl is as defined above.

The term “arylthio” as used herein represents a group —S-aryl wherein aryl is as defined above.

The term “aryloxy” as used herein represents a group —O-aryl wherein aryl is as defined above.

The term “arylsulfonyl” as used herein represents a group —(SO)₂-aryl wherein aryl is as defined above.

The term “arylamino” as used herein represents a group —NH-aryl wherein aryl is as defined above.

The term “heteroaroyl” as used herein represents a group —(CO)-heteroaryl wherein heteroaryl is as defined above.

The term “heteroaryloxy” as used herein represents a group —O-heteroaryl wherein heteroaryl is as defined above.

The term “heteroarylsulfonyl” as used herein represents a group —(SO)₂-heteroaryl wherein heteroaryl is as defined above.

The term “heteroarylamino” as used herein represents a group —NH-heteroaryl wherein heteroaryl is as defined above.

The term “C₁₋₅alkylC₃₋₇cycloalkyl” as used herein refers to a cycloalkyl group as defined above attached through an alkyl group as defined above having the indicated number of carbon atoms.

The term “C₁₋₅alkylC₃₋₇heterocycloalkyl” as used herein refers to a heterocycloalkyl group as defined above attached through an alkyl group as defined above having the indicated number of carbon atoms.

“Halogen” designates an atom selected from the group consisting of F, Cl, Br and I.

The structure A below designates a ring system with one or more substituents X, the possible number of X depends on the size and type of ring system. A is aryl, heteroaryl, cycloalkyl and heterocycloalkyl as defined above.

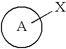

The structure B below designates a ring system with one or more substituents Y, the possible number of Y depends on the size and type of ring system. B is aryl, heteroaryl, cycloalkyl and heterocycloalkyl as defined above.

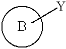

-L- is a linker having two free bonds; L is C₁₋₆-alkyl or C₂₋₆alkenyl, or a moiety selected from the group consisting of (wherein n is the same or different integer from 0 to 3)

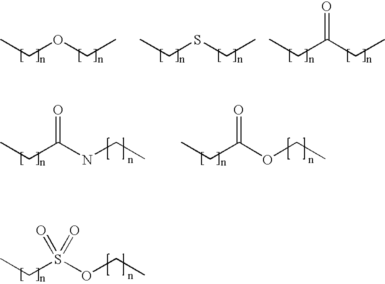

The terms “unsubstituted” or “substituted” as used herein means that the groups in question are optionally unsubstituted or substituted with one, two or three substituents independently of each other selected from the group consisting of C₁₋₆-alkyl, C₁₋₆alkoxy, C₁₋₆alkylthio, C₁₋₆alkylcarbonyl, C₁₋₆N-alkylamide, dialkylamino-C₁₋₆-alkyl, amide, hydroxy, carboxy, amino, nitro, halogen, trifluoromethyl, trifluoromethoxy, trifluoromethylthio and cyano. When the groups in question are substituted with more than one substituent the substituents may be the same or different.

The terms “amino acid”, “amino acid residue”, “natural amino acid” and “natural amino acid residue” as used herein all refer to the D- or L-isomers of the 20 standard amino acid residues: alanine (Ala), arginine (Arg), asparagine (Asn), aspartic acid (Asp), cysteine (Cys), glutamine (Gln), glutamic acid (Glu), glycine (Gly), histidine (His), isoleucine (Iie), leucine (Leu), lysine (Lys), methionine (Met), phenylalanine (Phe), proline (Pro), serine (Ser), threonine (Thr), tryptophan (Trp), tyrosine (Tyr) and valine (Val).

The terms “unnatural amino acid” and “non-natural amino acid residue” as used herein refer to non-standard or modified or unnatural amino acid residues. Examples of non-standard amino acid residues are 4-hydroxyproline, 6-N-methyl lysine, 2-aminoisobutyric acid, isovaline, and alpha-methyl serine. Examples of unnatural amino acid residues are pipecolic acid, thiazolidine carboxylic acid, dehydroproline, 3- and 4-methylproline, and 3,3-dimethylproline.

Certain of the above defined terms may occur more than once in the structural formulae, and upon such occurrence each term shall be defined independently of the other.

As used herein, the phrase “a functional group which can be converted to hydrogen in vivo” is intended to include any group that upon administering the present compounds to the subjects in need thereof can be converted to hydrogen enzymatically or by the acidic environment in the stomach. Non-limiting examples of such groups are acyl, carbamoyl, monoalkylated carbamoyl, dialkylated carbamoyl, alkoxycarbonyl, alkoxyalkyl groups and the like such as C₁₋₆alkylcarbonyl, aroyl, C₁₋₆alkylcarbamoyl (C₁₋₆N-alkylamide), di-C₁₋₆-alkyl-alkylcarbamoyl, C₁₋₆alkoxycarbonyl and C₁₋₆-alkoxy-C₁₋₆alkyl.

As used herein, the phrase “diseases and disorders related to dipeptidyl-peptidase I” is intended to include any disease or disorder in which an effect, preferably an inhibiting effect, on the dipeptidyl-peptidase I enzyme is beneficial.

The term “IC₅₀” as used herein denotes the concentration required for 50% inhibition of DPP-I in a binding assay.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

The Compounds

The present invention relates of compounds of the general formula (I) 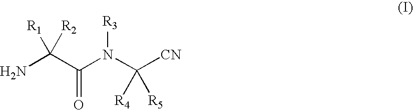
or a pharmaceutically acceptable salt or prodrug thereof, wherein
R₁ is hydrogen, C₁₋₆alkyl optionally substituted with a substituent selected from the group consisting of halogen, amino, hydroxy, cyano and C₁₋₃alkoxy; or C₂₋₆alkenyl, C₂₋₆alkynyl, C₁₋₆alkoxy, C₁₋₆alkylthio, C₁₋₆alkylcarbonyl, an unsubstituted or substituted C₃₋₁₀cycloalkyl group, an unsubstituted or substituted C₃₋₁₀cycloalkylcarbonyl group, an unsubstituted or substituted C₅₋₁₀cycloalkenyl group, an unsubstituted or substituted C₃₋₇heterocycloalkyl group, an unsubstituted or substituted C₁₋₆-alkylaryl group, an unsubstituted or substituted C₂₋₆alkenylaryl group, an unsubstituted or substituted C₁₋₆alkylheteroaryl group, an unsubstituted or substituted aryl group, an unsubstituted or substituted heteroaryl group, an unsubstituted or substituted aroyl group, an unsubstituted or substituted arylthio group, an unsubstituted or substituted aryloxy group, an unsubstituted or substituted arylsulfonyl group, an unsubstituted or substituted arylamino group, an unsubstituted or substituted heteroaroyl group, an unsubstituted or substituted heteroaryloxy group, an unsubstituted or substituted heteroarylsulfonyl group, an unsubstituted or substituted heteroarylamino group, an unsubstituted or substituted C₁₋₅alkylC₃₋₇cycloalkyl group or an unsubstituted or substituted C₁₋₅alkylC₃₋₇heterocycloalkyl group;
R₂ is hydrogen or C₁₋₆alkyl; or R₁ and R₂ together form an unsubstituted or substituted C₃₋₁₀cycloalkyl group or an unsubstituted or substituted C₃₋₇heterocycloalkyl group;
R₃ is hydrogen or C₁₋₆alkyl; or R₁ and R₃ together form an unsubstituted or substituted C₃₋₇heterocycloalkyl group;
R₄ is hydrogen, C₁₋₆alkyl, C₂₋₆alkenyl, C₂₋₆alkynyl, C₁₋₆alkoxy, C₁₋₆alkylthio, C₁₋₆alkylcarbonyl, C₁₋₆-alkylsulfonyl, an unsubstituted or substituted C₃₋₁₀cycloalkyl group, an unsubstituted or substituted C₃₋₁₀cycloalkylcarbonyl group, an unsubstituted or substituted C₅₋₁₀cycloalkenyl group, an unsubstituted or substituted C₃₋₇heterocycloalkyl group, an unsubstituted or substituted C₁₋₆-alkylaryl group, an unsubstituted or substituted C₂₋₆alkenylaryl group, an unsubstituted or substituted C₁₋₆-alkylheteroaryl group, an unsubstituted or substituted aryl group, an unsubstituted or substituted heteroaryl group, an unsubstituted or substituted aroyl group, an unsubstituted or substituted arylthio group, an unsubstituted or substituted aryloxy group, an unsubstituted or substituted arylsulfonyl group, an unsubstituted or substituted arylamino group, an unsubstituted or substituted heteroaroyl group, an unsubstituted or substituted heteroaryloxy group, an unsubstituted or substituted heteroarylsulfonyl group, an unsubstituted or substituted heteroarylamino group, an unsubstituted or substituted C₁₋₅alkylC₃₋₇cycloalkyl group or an unsubstituted, substituted C₁₋₅alkylC₃₋₇heterocycloalkyl group or a group of the formula: 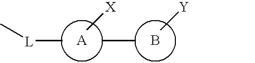
wherein A is a ring system with one or more substituents X, and A is selected from C₅₋₇cycloalkyl, C₅₋₇heterocycloalkyl, aryl and heteroaryl;
X being the same or different selected from hydrogen, Cl, Br, F, I, hydroxy, amino, cyano, trifluoromethyl, C₁₋₆-alkyl, C₁₋₆alkylthio or C₁₋₆-alkoxy;
B is a ring system with one ore more substituents Y, and B is selected from C₅₋₇cycloalkyl, C₅₋₇heterocycloalkyl, aryl and heteroaryl;
Y being the same or different selected from hydrogen, Cl, Br, F, 1, hydroxy, amino, cyano, trifluoromethyl, C₁₋₆-alkyl, C₁₋₆-alkylthio or C₁₋₆-alkoxy;
-L- is a linker, which is C₁₋₆-alkyl or C₂₋₆alkenyl, or a moiety selected from the group consisting of 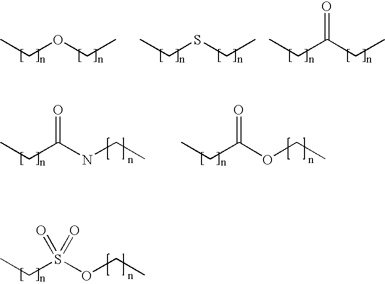
and, wherein the linker -L- may be attached via either of the two free bonds to the ring A;
n is the same or different integer selected from 0, 1, 2 and 3;
R₅ is hydrogen or C₁₋₆alkyl; or R₄ and R₅ together form an unsubstituted or substituted C₃₋₁₀cycloalkyl group or an unsubstituted or substituted C₃₋₇heterocycloalkyl group;
wherein a substituted group is substituted with one, two or three substituents independently selected from the group consisting of C₁₋₆-alkyl, C₁₋₆-alkoxy, C₁₋₆-alkylthio, C₁₋₆-alkylcarbonyl, C₁₋₆—N-alkylamide, dialkylamino-C₁₋₆-alkyl, amide, hydroxy, carboxy, amino, nitro, halogen, trifluoromethyl, trifluoromethoxy, trifluoromethylthio and cyano.

R₁ is typically selected from the group consisting of hydrogen, C₁₋₆-alkyl, an unsubstituted or substituted aryl, an unsubstituted or substituted C₁₋₆-alkylaryl group, an unsubstituted or substituted C₁₋₆-alkylheteroaryl group, or an unsubstituted or substituted C₃₋₁₀-cycloalkyl group.

In a specific embodiment R₁ is hydrogen, methyl, ethyl, n-propyl, isopropyl, n-butyl, isobutyl, sec-butyl, tert-butyl, phenyl, benzyl or cyclohexyl. Especially of interest are compounds wherein R₁ is hydrogen, methyl or ethyl.

R₁ and R₂ together may form an unsubstituted or substituted C₃₋₁₀cycloalkyl group or an unsubstituted or substituted C₃₋₇heterocycloalkyl group such as an unsubstituted or substituted cyclohexyl group.

R₁ and R₃ together may form an unsubstituted or substituted C₃₋₇heterocycloalkyl group such as a pyrrolidonyl or piperidonyl.

In specific embodiments R₂ is hydrogen and/or R₃ is hydrogen or methyl.

R₄ may be selected from the group consisting of hydrogen, C₁₋₆alkyl, an unsubstituted or substituted C₁₋₆alkylaryl group, an unsubstituted or substituted C₁₋₆alkenylaryl group and an unsubstituted or substituted C₁₋₆alkylheteroaryl group.

In interesting compounds according to the invention R₄ is hydrogen, unsubstituted or substituted benzyl, 2-phenylethyl, 3-phenylprop-2-ene, [1,1′-biphenyl-4-yl]methyl or [1,1′-biphenyl-2-yl]methyl.

In specific embodiments R₅ is hydrogen or R₄ and R₅ together form an unsubstituted or substituted C₃₋₁₀cycloalkyl group or an unsubstituted or substituted C₃₋₇heterocycloalkyl group.

In a particular embodiment at least one of R₄ and R₅ is hydrogen.

More specifically, a compound according to the invention may have the following structure 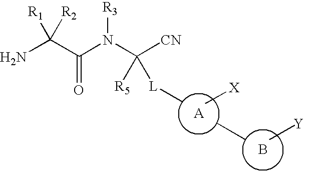
wherein R1, R2, R3, R5, A, B, X, Y and L are defined above.

Other interesting R₄ groups are

[1,1′-biphenyl-4-yl]methyl, [1,1′,2-methylbiphenyl-4-yl]methyl, [1,1′,3-methylbiphenyl-4-yl]methyl, [1,1′,2-hydroxybiphenyl-4-yl]methyl, [1,1′,3-hydroxybiphenyl-4-yl]methyl, [1,1′,2-methoxybiphenyl-4-yl]methyl, [1,1′,3-methoxybiphenyl-4-yl]methyl, [1,1′,2-methylthiobiphenyl-4-yl]methyl, [1,1′,3-methylthiobiphenyl-4-yl]methyl, [1,1′,2-cyanobiphenyl-4-yl]methyl, [1,1′,3-cyanobiphenyl-4-yl]methyl, [1,1′,2-aminobiphenyl-4-yl]methyl, [1,1′,3-aminobiphenyl-4-yl]methyl, [1,1′,2-fluorobiphenyl-4-yl]methyl, [1,1′,3-fluorobiphenyl-4-yl]methyl, [1,1′,2-chlorobiphenyl-4-yl]methyl, [1,1′,3-chlorobiphenyl-4-yl]methyl, [1,1′,2-bromobiphenyl-4-yl]methyl, [1,1′,3-bromobiphenyl-4-yl]methyl, [1,1′, 2′-fluorobiphenyl-4-yl]methyl, [1,1′,3′-fluorobiphenyl-4-yl]methyl, [1,1′,4′-fluorobiphenyl-4-yl]methyl, [1,1′,2′-chlorobiphenyl-4-yl]methyl, [1,1′,3′-chlorobiphenyl-4-yl]methyl, [1,1′,4′-chlorobiphenyl-4-yl]methyl, [1,1′,2′-bromobiphenyl-4-yl]methyl, [1,1′,3′-bromobiphenyl-4-yl]methyl, [1,1′,4′-bromobiphenyl-4-yl]methyl, [1,1′,2′-cyanobiphenyl-4-yl]methyl, [1,1′,3′-cyanobiphenyl-4-yl]methyl, [1,1′,4′-cyanobiphenyl-4-yl]methyl, [1,1′,4′-hydroxybiphenyl-4-yl]methyl, [1,1′,4′-aminobiphenyl-4-yl]methyl, [1,1′,4′-methoxybiphenyl-4-yl]methyl, [1,1′,4′-methylthiobiphenyl-4-yl]methyl, [1,1′,4′-trifluoromethylbiphenyl-4-yl]methyl, [1,1′,2-methyl-4′-fluorobiphenyl-4-yl]methyl, [1,1′,2-chloro-4′-cyanobiphenyl-4-yl]methyl, [1,1′,2-methoxy-3′-fluorobiphenyl-4-yl]methyl, [1,1′,2-hydroxy-2′-fluorobiphenyl-4-yl]methyl, [1,1′,3-amino-3′-methoxybiphenyl-4-yl]methyl, [1,1′,2-fluoro-4′-fluorobiphenyl-4-yl]methyl [2-phenyl-1,3-thiazol-4-yl]methyl, [5-phenylpyridin-3-yl]methyl, [3-pyrimidin-5-ylphenyl]methyl, [3-pyridin-2-ylphenyl]methyl, [3-pyridin-4-ylphenyl]methyl, [3-(1H-indol-6-yl)phenyl]methyl, [1-(2-fluorophenyl)piperidin-4-yl]methyl, [3-fluoro-4-(1-piperidinyl)phenyl]methyl, [1,1′-biphenyl-4-yl]ethyl, [1,1′,2-methylbiphenyl-4-yl]ethyl, [1,1′,3-methylbiphenyl-4-yl]ethyl, [1,1′,2-hydroxybiphenyl-4-yl]ethyl, [1,1′,3-hydroxybiphenyl-4-yl]ethyl, [1,1′,2-methoxybiphenyl-4-yl]ethyl, [1,1′,3-methoxybiphenyl-4-yl]ethyl, [1,1′,2-methylthiobiphenyl-4-yl]ethyl, [1,1′,3-methylthiobiphenyl-4-yl]ethyl, [1,1′,2-cyanobiphenyl-4-yl]ethyl, [1,1′,3-cyanobiphenyl-4-yl]ethyl, [1,1′,2-aminobiphenyl-4-yl]ethyl, [1,1′,3-aminobiphenyl-4-yl]ethyl, [1,1′,2-fluorobiphenyl-4-yl]ethyl, [1,1′,3-fluorobiphenyl-4-yl]ethyl, [1,1′,2-chlorobiphenyl-4-yl]ethyl, [1,1′,3-chlorobiphenyl-4-yl]ethyl, [1,1′,2-bromobiphenyl-4-yl]ethyl, [1,1′,3-bromobiphenyl-4-yl]ethyl, [1,1′,2′-fluorobiphenyl-4-yl]ethyl, [1,1′,3′-fluorobiphenyl-4-yl]ethyl, [1,1′,4′-fluorobiphenyl-4-yl]ethyl, [1,1′,2′-chlorobiphenyl-4-yl]ethyl, [1,1′,3′-chlorobiphenyl-4-yl]ethyl, [1,1′,4′-chlorobiphenyl-4-yl]ethyl, [1,1′,2′-bromobiphenyl-4-yl]ethyl, [1,1′,3′-bromobiphenyl-4-yl]ethyl, [1,1′,4′-bromobiphenyl-4-yl]ethyl, [1,1′,2′-cyanobiphenyl-4-yl]ethyl, [1,1′,3′-cyanobiphenyl-4-yl]ethyl, [1,1′,4′-cyanobiphenyl-4-yl]ethyl, [1,1′,4′-trifluoromethylbiphenyl-4-yl]ethyl, [1,1′,2-methyl-4′-fluorobiphenyl-4-yl]ethyl, [1,1′,2-chloro-4′-cyanobiphenyl-4-yl]ethyl, [1,1′,2-methoxy-3′-fluorobiphenyl-4-yl]ethyl, [1,1′,2-hydroxy-2′-fluorobiphenyl-4-yl]ethyl, [1,1′,3-amino-3′-methoxybiphenyl-4-yl]ethyl, [2-phenyl-1,3-thiazol-4-yl]ethyl, [5-phenylpyridin-3-yl]ethyl, [3-pyrimidin-5-ylphenyl]ethyl, [3-pyridin-2-ylphenyl]ethyl, [3-pyridin-4-ylphenyl]ethyl, [3-(1H-indol-6-yl)phenyl]ethyl, [1-(2-fluorophenyl)piperidin-4-yl]ethyl, [3-fluoro-4-(1-piperidinyl)phenyl]ethyl, [1,1′-biphenyl-4-yl]methyloxymethyl, [1,1′,4′-fluorobiphenyl-4-yl]methyloxymethyl, [1,1′-biphenyl-4-yl]methylthiomethyl, [1,1′,4′-fluorobiphenyl-4-yl]methylthiomethyl, [1,1′-biphenyl-4-yl]ethylenyl or [1,1′,4′-fluorobiphenyl-4-yl]ethylenyl.

Preferred compounds of the invention are:
N-(2S-2-amino-3-phenylpropionyl)-aminoacetonitrile;
(2S)—N-[(2S)-2-aminobutanoyl]-2-amino-3-phenylpropionitrile; (2S)—N-Methyl-N-[(2S)-2-aminobutanoyl]-2-amino-3-phenylpropionitrile;
(2S)—N-[(2S)-2-aminobutanoyl]-2-amino-3-(p-chlorophenyl)propionitrile;
(2S)—N-[(2S)-2-aminobutanoyl]-2-amino-3-(1,1′-biphenyl-4-yl)propionitrile;
(2S)-(4Z)-N-[(2S)-2-aminobutanoyl]-2-amino-5-phenyl-pent-4-ene-nitrile;
(2S)—N-[(2S)-2-aminobutanoyl]-2-amino-4-phenylbutyronitrile; and
(2S)—N-[(2S)-3-phenylaminopropanoyl]-2-amino-3-phenylpropionitrile.

The compounds of the invention may exist as geometric isomers or optical isomers or stereoisomers as well as tautomers. Accordingly, the invention includes all geometric isomers and tautomers including mixtures and racemic mixtures of these and a pharmaceutically acceptable salt thereof, especially all R- and S-isomers.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates, which the present compounds are able to form.

The acid addition salts may be obtained as the direct products of compound synthesis.

In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs such as bioreversible derivatives formed by reaction of the N-terminal with a suitable transport group resulting e.g. in amides, carbamates etc. of the present compounds, which on administration undergo chemical conversion by metabolic processes before becoming active pharmacological substances. In general, such prodrugs will be functional derivatives of the present compounds, which are readily convertible in vivo into the required compound of the Formula I. Prodrugs are any covalently bonded compounds, which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The present invention includes all complexes of the compounds of this invention.

The meaning of any substituent at any one occurrence in Formula I or any subformula thereof is independent of its meaning, or any other substituent's meaning, at any other occurrence, unless specified otherwise.

In a preferred embodiment of this invention, the compounds of Formula I exhibit an IC₅₀ value of less than 500 μM, preferably less than 100 μM, more preferably less than 50 μM, even more preferably less than 1 μM, especially less than 500 nM, particularly less than 100 nM, when subjected to a human dipeptidyl dipeptidase-I assay such as the assay disclosed herein.

Methods of Preparation

The compounds of the present invention may be prepared by the methods set forth in scheme 1-3 below.

A list of the used abbreviations is given below under Materials and Methods.

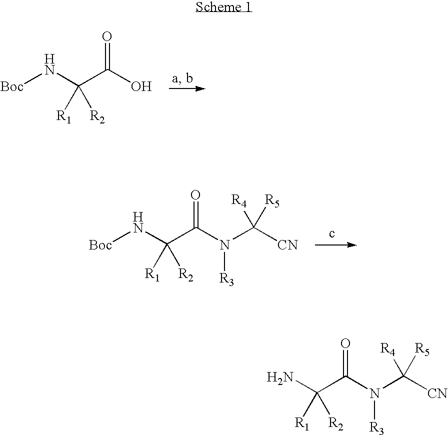

Reagents and conditions: a) CDI, THF; b) R₃NHCHR₄R₅CN, THF; c) TFA The appropriate Boc-protected amino acid derivative is activated by reaction with CDI in a solvent such as THF. Subsequent addition of the free base of the appropriate amino acid nitrite derivative gives the Boc protected dipeptidyl nitrite, which is deprotected with TFA. The crude products are then purified by HPLC.

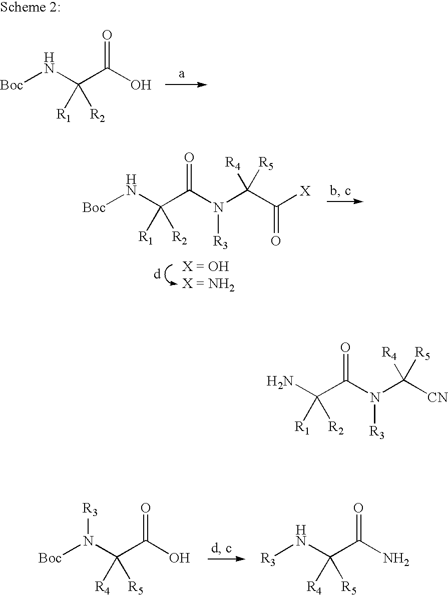

Reagents and conditions: a) CDI, THF; then R₃HNC(R₄R₅)COOH, base or R₃HNC(R₄R₅)CONH₂*HCl, base; b) POCl₃, imidazole, pyridine, −40° C.→room temperature; c) TFA; d) CDI, THF; then NH₃ in 2-propanol.

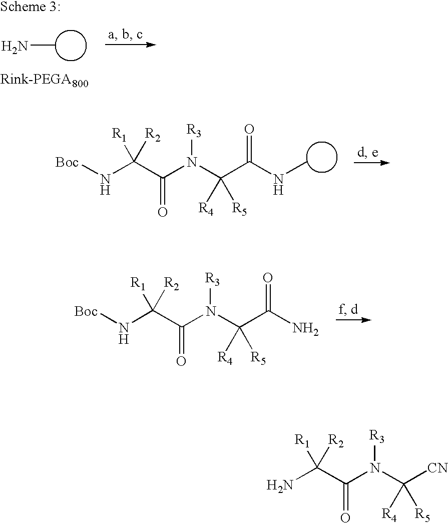

Reagents and conditions: a) Fmoc(R₃)NC(R₄R₅)COOH, TBTU, NEM, DMF; b) 20% piperidine, DMF; c) BocNHC(R₁R₂)COOH, TBTU, NEM, DMF; d) TFA; e) Boc₂O, NEM; f) POCl₃, imidazole, pyridine, −40° C.→room temperature.

The compounds of the invention can also be prepared according to scheme 2, in which the nitrile is formed at the last step by dehydration of the protected dipeptide amide, using an appropriate dehydrating agent, such as POCl₃/imidazole in pyridine. Alternatively, the dipeptide nitrites can be prepared according to scheme 3 via assembly of the protected dipeptide amide precursor on solid phase, using an acid-labile linker, such as the Rink amide linker.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, III., 1984, are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W. PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

Pharmaceutical Compositions

In one aspect of this invention, there is provided a pharmaceutical composition comprising, as an active ingredient, a compound of the present invention together with a pharmaceutically acceptable carrier or diluent. This composition may be in unit dosage form and may comprise from about 0.05 to about 100 mg, preferably from about 0.1 to about 50 mg, of the compound of the invention or a pharmaceutically acceptable salt or ester thereof. The composition of the invention may be used for oral, nasal, transdermal, pulmonal or parenteral administration. It is contemplated that the pharmaceutical composition of the invention is useful for treatment of inflammation, type2 diabetes, asthma, severe influenza, respiratory syncytial virus infection, CD8 T cell inhibition, inflammatory bowel diseases, psoriasis, atopic dermatitis, Papillon Lefevre syndrome, Haim Munk syndrome, gum disease, periodontitis, rheumatoid arthritis, Huntington's disease, Chagas' disease, Alzheimer's disease, and/or for application in target cell apoptosis.

The compounds of the invention may be administered alone or in combination with pharmaceutically acceptable carriers, diluents or excipients, in either single or multiple doses. Accordingly, the compounds of Formula I may be used in the manufacture of a medicament. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19.sup.th Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention. Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from about 1 μg to about 1000 mg such as, e.g., from about 10 μg to about 500 mg, from about 0.05 to about 100 mg or from about 0.1 to about 50 mg, of the active substance.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the Formula (I) contains a free base such salts are prepared in a conventional manner by treating a solution or suspension of a free base of the Formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the Formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the Formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet, which may be prepared by conventional tabletting techniques, may contain:

Core:

| | | |
|---|---|---|
| Active compound (free compound or salt) | 5.0 | mg |
| Lactosum Ph. Eur. | 67.8 | mg |
| Cellulose, microcryst. (Avicel) | 31.4 | mg |
| Amberlite | 1.0 | mg |

| | |
|---|---|
| Magnesii stearas | q.s. |

Coating:

| | | |
|---|---|---|
| Hydroxypropyl methylcellulose approx. | 9 | mg |
| Acylated monoglyceride approx. | 0.9 | mg |

If desired, the pharmaceutical composition of the invention may comprise the compound of the Formula (I) in combination with further pharmacologically active substances such as those described in the foregoing.

Use of the Invention

The compounds of Formula I are useful as protease inhibitors, particularly as inhibitors of cysteine and serine proteases, more particularly as inhibitors of cysteine proteases, even more particularly as inhibitors of cysteine proteases of the papain superfamily, yet more particularly as inhibitors of DPP-I. The present invention provides useful compositions and formulations of said compounds, including pharmaceutical compositions and formulations of said compounds.

The compounds of the present invention may especially be useful for the treatment or prevention of diseases such as inflammation, type2 diabetes and similar diseases involving a protease. The present compounds are especially useful for treating diseases in which cysteine proteases are implicated and especially diseases in which dipeptidyl peptidase-I is implicated, most particularly mast cell inflammatory mediated diseases. Examples of diseases in which dipeptidyl peptidase-I is implicated are: inflammation, type2 diabetes, asthma, severe influenza, respiratory syncytial virus infection, CD8 T cell inhibition, inflammatory bowel diseases, psoriasis, atopic dermatitis, Papillon Lefevre syndrome, Haim Munk syndrome, gum disease, periodontitis, rheumatoid arthritis, Huntington's disease, Chagas' disease, Alzheimer's disease, sepsis as well as in target cell apoptosis.

Accordingly, in one aspect the present invention relates to a method for the treatment of ailments, the method comprising administering to a subject in need thereof an effective amount of a compound or a composition of this invention. It is contemplated that an effective amount of a compound or a composition of this invention corresponds to an amount of active ingredient, i.e. active compound or a pharmaceutically acceptable salt or ester thereof, in the range of from about 1 μg to about 1000 mg such as, e.g., from about 10 μg to about 500 mg, from about 0.05 to about 100 mg or from about 0.1 to about 50 mg per day.

In yet another aspect, the present invention relates to use of a compound of this invention for the preparation of a medicament, preferably a medicament for treatment of inflammation, type2 diabetes, asthma, inflammatory bowel diseases, psoriasis, atopic dermatitis, Papillon Lefevre syndrome, Haim Munk syndrome, gum disease, periodontitis, arthritis, Huntington's disease, Chagas' disease, Alzheimer's disease, sepsis or for application in target cell apoptosis.

For acute therapy, parenteral administration of a compound of Formula I is preferred. An intravenous infusion of the compound in 5% dextrose in water or normal saline, or a similar formulation with suitable excipients, is most effective, although an intramuscular bone injection is also useful. Typically, the parenteral dose will be about 0.01 to about 100 mg/kg; preferably between 0.1 and 20 mg/kg, in a manner to maintain the concentration of drug in the plasma at a concentration effective to inhibit dipeptidyl dipeptidase-I (cathepsin C). The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

The compounds of the present invention fully or partly inhibit dipeptidyl-peptidase I, and are thus useful for the treatment and/or prevention of a wide variety of conditions and disorders in which inhibition of DPP-I is beneficial, especially in which selective inhibition of DPP-I is advantageous.

Accordingly, in another aspect the present invention relates to a compound of the general Formula (I) or any optical or geometric isomer or tautomeric form thereof including mixtures of these or a pharmaceutically acceptable salt thereof for use as a pharmaceutical composition.

The invention also relates to pharmaceutical compositions comprising, as an active ingredient, at least one compound of the Formula (I) or any optical or geometric isomer or tautomeric form thereof including mixtures of these or a pharmaceutically acceptable salt thereof together with one or more pharmaceutically acceptable carriers or diluents.

Furthermore, the invention also relates to the use of the compounds and compositions of the present invention to modulate DPP-I levels in a subject (e.g., human) in need thereof in an amount effective to modulate DPP-I levels. In a preferred embodiment, the compound or composition inhibits DPP-I.

In the following synthetic example, all of the starting materials were obtained from commercial sources unless otherwise indicated. Without further elaboration, it is believed that one skilled in the art can, using the preceding description, utilize the present invention to its fullest extent. These examples are given to illustrate the invention, not to limit its scope.

### EXAMPLES

Materials and Methods

Temperature is in degrees Centigrade (° C.). Experiments were conducted at room temperature (20° C.), unless otherwise noted. All solvents were HPLC grade. Anhydrous solvents were obtained by storing over 4 Å activated molecular sieves. Unless otherwise noted starting materials were purchased from commercial suppliers and used without further purification.

NMR data were acquired on a Bruker Advance DRX 250. CDCl₃ is deuteriochloroform, DMSO-d₆ is hexadeuteriodimethylsulfoxide, D₂O is deuteriooxide, and CD₃OD is tetradeuteriomethanol. Abbreviation for NMR data are as follows: s=singlet, d=doublet, t=triplet, q=quartet, m=multiplet, dd=.doublet of doublets, dt=doublet of triplets, td=triplet of doublets, app=apparent, br=broad. Chemical shifts are reported in ppm, relative to internal solvent peaks (2.49 for DMSO-d₆, 7.25 for CDCl₃, 4.75 for D₂O, 3.35 for CD₃OD). Coupling constants J are reported in Hz. ES-MS spectra were obtained on a Micromass Quattro micro™ instrument in the positive mode unless otherwise noted. Analytical HPLC was performed on a Gilson system (UV/VIS-155 detector at 215 and 254 nm, 402 syringe pump, 819 injection module, valvemate 35, 864 degasser, 233 XL on-line column switching module, and a Zorbax 300SB RP-18 column, 4.6×50 mm with a 322 pump). Eluents A (0.1% TFA in water) and B (1% TFA in acetonitrile) were used in a linear gradient (0% B→100% B in 7 min.). Purity (given in parentheses) is at 215 nm. Preparative HPLC was performed on the same Gilson system, using a Zorbax 300SB RP-18, 21.2 mm×25 cm column, with a flow of 15 mL/min.

Abbreviations

AFC 7-Amino-trifluoromethyl coumarin
CDI 1,1′-Carbonyldiimidazole
DCM Dichloromethane
DE Diethylether
DIPEA N,N-Diisopropylethylamine
DMF N,N-Dimethylformamide
DMSO Dimethyl sulfoxide
EA Ethyl acetate
EDTA Ethylenediaminetetraacetic acid
NEM N-Ethylmorpholine
TFA Trifluoroacetic acid
THF Tetrahydrofurane
TIPS Triisopropylsilane

### Example 1

### N-(2S-2-Amino-3-phenylpropionyl)-aminoacetonitrile

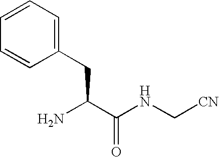

Boc-Phe-OH (50 mg, 0.19 mmol) and CDI (33.5 mg, 0.21 mmol) were dissolved in dry THF (2 mL). After 30 min. aminoacetonitrile hydrogen sulfate (29 mg, 0.19 mmol) and DIPEA (66 μL, 0.38 mmol) were added. The resulting solution was stirred at rt for 18 h, then poured into 10% aq. citric acid (25 mL), and extracted with EA (×2). The combined organics were washed with brine (×1), dried (MgSO₄) and evaporated to give a clear oil. The Boc group was then removed by treatment with 95% aq. TFA (2 mL) for 1 h, followed by HPLC purification of the crude product. The title compound was obtained as a white solid. Yield: 33 mg (86%); HPLC: Rt=2.59 min. (>99%); ¹H-NMR (DMSO-d₆, 250 MHz) δ 9.31-9.27 (t, J=5.5, 1H), 8.39 (br, 2H), 7.39-7.21 (m, 5H), 4.21-4.19 (d, J=5.5, 2H), 4.09-3.97 (m, 1H), 3.13-3.04 (m, 2H); ES-MS: mass calcd for C₁₁H₁₄N₃O 204.1 (MH⁺). Found m/z 204.1.

### Example 2

### (2S)—N-[(2S)-2-aminobutanoyl]-2-amino-3-phenylpropionitrile

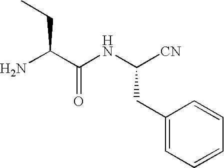

Nα-Boc-L-aminobutyric acid (254 mg, 1.25 mmol) and CDI (224 mg, 1.38 mmol) were stirred in dry THF (10 mL) for 30 min. Then, H-Phe-NH2*HCl (250 mg, 1.25 mmol) and DIPEA (214 μL, 1.25 mmol) in dry THF (20 mL) were added and the resulting suspension was stirred o.n. at rt. The volume was reduced to half and the suspension was stirred for another 18 h after which it was taken to dryness. The residue was suspended in EA, washed with 10% aq. citric acid (×1), sat. NaHCO₃ (×1) and brine (×1), dried (MgSO₄) and concentrated in vacuo yielding 440 mg solid. The crude amide (396 mg, 1.12 mmol), which was >95% pure by HPLC, was dissolved in dry pyridine (5 mL) along with imidazole (99 mg, 1.46 mmol). The solution was cooled to −45° C. and POCl₃(165 μL, 1.8 mmol) was added. The resulting yellow-orange solution was stirred at −45° C. for 1 h, then taken to rt and stirred for 1 h. Work-up as above to give the crude nitrile as a dark residue (260 mg) with a crude purity of >95% by HPLC. The Boc group was removed by treatment with TFA:TIPS 95:5 for 30 min. followed by evaporation to give a brown oil. Purification by HPLC gave the title compound as a white solid. Yield: 124 mg (48%); HPLC: Rₜ=3.19 min. (>99%); ¹H-NMR (DMSO-d₆, 250 MHz) δ (isomers in a 3:1 ratio; isomeric signal given in parentheses) 9.36-9.33 (d, J=7.3, 1H, not present in D₂O), 8.21 (br, 2H, not present in D₂O), 7.36-7.26 (m, 5H), 5.13-4.97 (m, 1H), 3.79-3.72 (m, 1H), 3.26-3.05 (m, 2H), 1.83-1.70 (m, 2H) (isomer:1.59-1.53), 0.91-0.85 (t, J=7.4, 3H)(isomer: 0.59-0.53); ¹³C-NMR (DMSO-d₆, 250 MHz) δ 9.0(8.6), 24.6(24.4), 37.1(37.6), 42.4(41.6), 53.3(53.3), 118.9(119.3), 127.6, 128.8, 129.7, 135.6, 169.3(168.9); ES-MS: mass calcd for C₁₃H₁₈N₃O 232.1 (MH⁺). Found m/z 232.2.

### Example 3

### (2S)—N-Methyl-N-[(2S)-2-aminobuta noyl]-2-amino-3-phenylpropionitrile

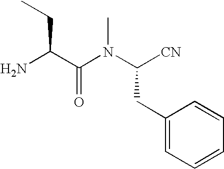

Fmoc-Rink-PEGA800 resin (0.8 g, L=0.4 mmol/g, 0.32 mmol) was Fmoc deprotected and washed with DMF (×5). Fmoc-N-Me-Phe-OH (385 mg, 0.96 mmol) was coupled via TBTU (295 mg, 0.92 mmol) with NEM (365 μL, 1.28 mmol) in dry DMF. The resin was washed with DMF (×5), Fmoc deprotected and washed with DMF (×5). Nα-Boc-L-aminobutyric acid (195 mg, 0.96 mmol) was then similarly coupled via TBTU. Wash with DMF (×5), DCM (×2) and CH₃CN (×2) followed by lyophilization. The dipeptide amide was cleaved from the resin by treatment with TFA:TIPS 95:5 for 30 min. followed by wash with TFA:TIPS 95:5 (×5). The combined fractions were concentrated in vacuo and the resulting oil was lyophilized. The amino group was re-protected with Boc by reaction with Boc₂O (139 mg, 0.32 mmol) and DIPEA (53 μL, 0.38 mmol) in dry CH₃CN (2 mL) at rt o.n. Purification by HPLC gave the Boc-protected dipeptide amide as a white residue (18 mg, 15%). The amide was dehydrated with POCl₃/pyridine/imidazole (15 μL/1.2 mL/4.7 mg) and subsequently Boc-deprotected as described in example 2. Purification by HPLC gave the title compound as a white solid. Yield: 6 mg (8%); HPLC: Rt=3.50 min. (>99%); ¹H-NMR (CD₃OD, 250 MHz) 6 (isomers in a 5:1 ratio; isomeric signal given in parentheses) 7.41-7.32 (m, 5H), 5.62-5.54 (m, 1H), 4.41-4.32 (m, 1H), 3.32-3.15 (m, 2H), 3.14 (br s, 3H), 1.93-1.83 (m, 2H), 1.11-1.04 (t, J=7.3, 3H) (isomer: 0.72-0.65); ES-MS: mass calcd for C₁₄H₂₀N₃O 246.2 (MH⁺). Found m/z 246.2.

### Example 4

### (2S)—N-[(2S)-2-aminobutanoyl]-2-amino-3-(p-chlorophenyl)propionitrile

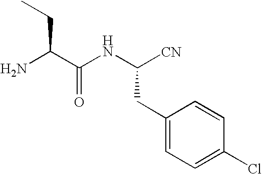

Starting from Fmoc-Rink-PEGA800 resin (1 g, L=0.48 mmol/g, 0.48 mmol) using the procedure for example 3 the following changes were applied: Fmoc-p-Cl-Phe-OH (608 mg, 1.44 mmol) was used instead of Fmoc-N-Me-Phe-OH, and Nα-Fmoc-L-aminobutyric acid (469 mg, 1.44 mmol) was used instead of Nα-Boc-L-aminobutyric acid. The Fmoc group was removed prior to cleavage from the resin. White solid. Yield: 11 mg (9%); HPLC: Rt=3.79 min. (>99%); ¹H-NMR (CD₃OD, 250 MHz) 6 (isomers in a 6:1 ratio; isomeric signal given in parentheses) 7.52-7.41 (m, 4H), 5.25-5.18 (m, 1H), 3.96-3.88 (m, 1H), 3.36-3.28 (m, 2H), 2.09-1.98 (m, 2H), 1.20-1.13 (t, J=7.5, 3H)(isomer: 0.93-0.85); ES-MS: mass calcd for C₁₃H₁₇ClN₃O 266.1 (MH⁺). Found m/z 266.3

### Example 5

### (2S)—N-[(2S)-2-aminobutanoyl]-2-amino-3-(1,1′-biphenyl-4-yl)propionitrile

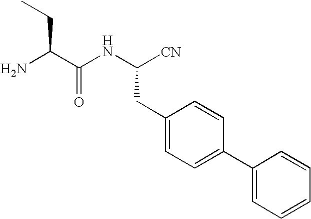

Starting from Fmoc-Rink-PEGA800 resin (1 g, L=0.39 mmol/g, 0.39 mmol) using the procedure for example 3 the following changes were applied: Fmoc-p-Ph-Phe-OH (542 mg, 1.17 mmol) was used instead of Fmoc-N-Me-Phe-OH. White solid. Yield: 11 mg (9%); HPLC: Rt=4.58 min. (>99%); ¹H-NMR (CD₃OD, 250 MHz) δ (isomers in a 11:1 ratio; isomeric signal given in parentheses) 7.75-7.45 (m, 9H), 5.25-5.19 (m, 1H), 3.93-3.86 (t, J=6.3, 1H), 3.37-3.31 (m, 2H, partially hidden under CD₃OD signal), 2.08-1.97 (m, 2H), 1.17-1.11 (t, J=7.3, 3H)(isomer: 0.94-0.91); ES-MS: mass calcd for C₁₉H₂₂N₃O 308.2 (MH⁺). Found m/z 308.2.

### Example 6

### (2S)-(4Z)-N-[(2S)-2-aminobutanoyl]-2-amino-5-phenyl-pent-4-ene-nitrile

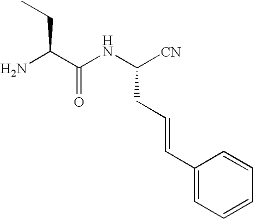

Starting from Fmoc-Rink-PEGA800 resin (1 g, L=0.30 mmol/g, 0.30 mmol) using the procedure for example 3 the following changes were applied: Fmoc-Styrylalanine (372 mg, 0.9 mmol) was used instead of Fmoc-N-Me-Phe-OH. White solid. Yield: 16 mg (21%); HPLC: Rt=4.13 min. (>99%); ¹H-NMR (CD₃OD, 250 MHz) δ (isomers in a 7:1 ratio; isomeric signal given in parentheses) 7.61-7.25 (m, 5H), 6.81-6.72 (m, 1H), 6.46-6.20 (m, 1H), 5.13-5.08 (t, J=7.2, 1H), 4.03-3.92 (t, J=6.3, 1H), 3.00-2.84 (t, J=7.2 2H), 2.15-1.94 (m, 2H), 1.20-1.14 (t, J=7.4, 3H)(isomer: 1.08-1.02); ES-MS: mass calcd for C₁₅H₂₀N₃O 258.2 (MH⁺). Found m/z 258.1.

### Example 7

### (2S)—N-[(2S)-2-aminobutanoyl]-2-amino-4-phenylbutyronitrile

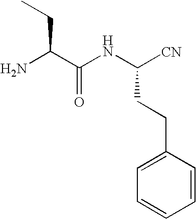

Starting from Fmoc-Rink-PEGA800 resin (0.8 g, L=0.40 mmol/g, 0.32 mmol) using the procedure for example 3 the following changes were applied: Fmoc-Homophenylalanine (385 mg, 0.96 mmol) was used instead of Fmoc-N-Me-Phe-OH, and Nα-Fmoc-L-aminobutyric acid (312 mg, 0.96 mmol) was used instead of Nα-Boc-L-aminobutyric acid. The Fmoc group was removed after formation of the nitrile, using piperidine (340 μL, 20 eq.) in DCM (1 mL). Purified by HPLC. White solid. Yield: 11 mg (14%); HPLC: Rt=3.59 min. (>99%); ¹H-NMR (CD₃OD, 250 MHz) δ 7.45-7.29 (m, 5H), 4.90-4.84 (t, J=7.6, 1H), 3.98-3.93 (t, J=6.3, 1H), 2.95-2.77 (m, 2H), 2.36-1.95 (m, 4H), 1.19-1.13 (t, J=7.5, 3H); ES-MS: mass calcd for C₁₄H₂₀N₃O 246.2 (MH⁺). Found m/z 246.2.

### Example 8

### (2S)—N-[(2S)-3-phenylaminopropanoyl]-2-amino-3-phenylpropionitrile

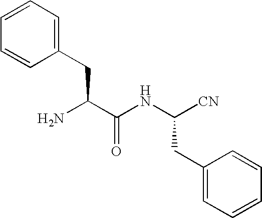

Following the procedure for example 2, using Boc-Phe-OH (332 mg, 1.25 mmol) instead of Nα-Boc-L-aminobutyric acid gave the title product (major isomer): White solid. Yield: 87 mg (24%); HPLC: Rt=3.82 min. (>98%); ¹H-NMR (CD₃OD, 250 MHz) δ 7.43-7.23 (m, 10H), 5.06-4.96 (t, J=7.7, 1H), 4.09-4.03 (t, J=6.7, 1H), 3.25-3.12 (m, 4H); ES-MS: mass calcd for C₁₈H₂₀N₃O 294.2 (MH⁺). Found m/z 294.2.

Biological Assays

The compounds of this invention may be tested in one of several biological assays to determine the concentration of compound, which is required to have the desired pharmacological effect.

Human dipeptidyl Peptidase I (DPP-I) Assay

Using this assay, the IC₅₀ value of a compound of the invention as a DPP-I inhibitor was determined using an AFC substrate.

Assay Buffer (pH 6.0):

100 mM sodium phosphate (8.9 g Na₂HPO₄; M=177.99), 150 mM KCl (5.6 g KCl; M=74.6) and 1.5 mM EDTA (279 mg EDTA; M=372.2) was dissolved in 500 mL H₂O, and pH was adjusted to 6.0. Cysteine*HCl (Sigma C-1276; M=157.6), 1 mg/mL assay buffer, corresponding to 6 mM, was added to the solution for activation of the DPP-1 enzyme.

Substrate:

Gly-Phe-AFC*TFA (Enzyme Systems Products AFC-033) was used as the substrate for determination of IC₅₀ values. Kₘ was 270 μM. The substrate was solubilized in DMSO to give a 7.5 mM stock solution (2.2 mg of substrate was added to 0.5 mL DMSO).

DPP-I:

Human DPP-I (hDDP-I; obtained from UniZyme A/S, DK-2970Hørsholm, Denmark) was stored at −20° C. in a buffer containing 2.5 mM Na-phosphate, 150 mM NaCl, 2 mM cysteamine, 50% glycerol, pH 7.0 at a concentration of 2.5 mg/mL. This stock solution was diluted 200 times in the assay buffer.

Assay Conditions:

The assay was performed in 96-well plates. Assay buffer (230 μL) was added to the well, followed by 10 μL of diluted DPP-I, corresponding to 9.1 nM in the assay. Then 5 μL of either DMSO (control) or test substance in varying concentrations was added, and the solution was mixed. The plate was incubated at 37° C. for 10 minutes, followed by addition of 5 μL of 7.5 mM substrate (corresponding to 150 μM in the assay). The excitation wavelength was 400 nm, and the emission was measured at 505 nm for 10 minutes at 37° C. Each measurement was made in duplicate. In the software (SOFTmax Pro) used for data collection from the fluorometer (Molecular Devices: Gemini XS), it was ensured that the measured slopes were linear (R²>0.99). Data were exported to GraphPad Prism and nonlinear regression was performed using the option Sigmoidal dose-response (variable slope).

Human Liver Cathepsin B Assay

Using this assay, the selectivity of a compound of the invention for hDPP-I over human cathepsin B was determined, using a fluorogenic substrate.

Assay Buffer (pH 6.1):

0.1 M MES buffer (1.95 g; M=195.2) and 1 mM EDTA (37 mg; M=372.2) were mixed in 100 mL H₂O and pH adjusted to 6.1. DTT (10 μL, 0.5 M) was added for activation of the enzyme (corresponding to 5 mM).

Cathepsin B:

Human liver cathepsin B (Enzyme System Products, CAT-B; stock 25 μg/54 μL=463 ng/μL). One aliquot was diluted to a concentration of 40 ng/μL by adding 53 μL assay buffer (without DTT). Just prior to experiment, two more dilution steps were performed: 4 ng/μL: 5 μL (40 ng/μL)+45 μL buffer (without DTT) 0.1 ng/μL: 5 μL (4 ng/μL)+285 μL buffer (without DTT)

Substrate:

Boc-Leu-Arg-Arg-AFC.2TFA (Enzyme System Products AFC113). Stock solution made (20 mM; 15.1 mg dissolved in 1 ml DMSO). Diluted further in H₂O to 10 mM. Kₘ for this substrate has been determined to be 600 μM.

Assay Conditions:

The assay was performed in 96-well plates. 84 μL assay buffer was added to the well followed by 10 μL 1% DMSO in assay buffer (control) or a compound of the invention (10 μM in the assay). Then 10 μL (0.1 ng/μL, corresponding to 1 ng in assay) enzyme was added, and 5 min. later 6 μL substrate (10 mM, corresponding to 600 μM in the assay) was added. The excitation wavelength was 400 nm, and the emission was measured at 505 nm for 10-20 minutes at 37° C. Each measurement was made in duplicate. In the software (SOFTmax Pro) used for data collection from the fluorometer (Molecular Devices: Gemini XS), it was ensured that the measured slopes were linear (R²>0.99). Data were exported to GraphPad Prism and nonlinear regression was performed using the option Sigmoidal dose-response (variable slope).

Human Liver Cathepsin H Assay

Using this assay, the selectivity of a compound of the invention for hDPP-I over human cathepsin H was determined, using a fluorogenic substrate.

Assay Buffer (pH 6.0):

50 mM sodium phosphate (0.89 g Na₂HPO₄; M=177.99), 2.0 mM EDTA (74 mg EDTA; M=372.2), 0.012% Triton-X (390 μL 3%) dissolved in 100 mL H₂O and pH adjusted to 6.0.

Cathepsin H:

Human liver cathepsin H (Enzyme System Products, Cath-1; 25 μg) was solubilized in 60 μL enzyme buffer (giving a stock with a concentration of 417 ng/μL). Enzyme stock (5 μL) was diluted with 1245 μL enzyme buffer. Prior to running experiment 1 μL 0.5 M DTT/100 μL enzyme solution was added. Incubated for 5 minutes on ice, then added to reaction mixture.

Substrate:

ARG-AFC*2HBR (Enzyme System Products AF002; 10.6 mg) was dissolved in 1 mL DMSO, giving a 20 mM solution. Km for this substrate has been determined to be 27 μM

Assay Conditions:

The assay was performed in 96-well plates. 50 μL assay buffer was added to the well followed by 25 μL reference inhibitor (cystatin; stock 1 mg/mL, diluted with assay buffer to give 10 nM in assay) or a compound of the invention (diluted with assay buffer to give 10 μM in the assay). Then 25 μL (40 ng) enzyme was added, and 1 min. later (at 37° C.), 100 μL ARG-AFC substrate (15 μM in the assay) was added. The excitation wavelength was 400 nm, and the emission was measured at 505 nm for 10-20 minutes at 37° C. Each measurement was made in duplicate. In the software (SOFTmax Pro) used for data collection from the fluorometer (Molecular Devices: Gemini XS), it was ensured that the measured slopes were linear (R²>0.99). Data were exported to GraphPad Prism and nonlinear regression was performed using the option Sigmoidal dose-response (variable slope).

Human Liver Cathepsin L Assay

Using this assay, the selectivity of a compound of the invention for hDPP-I over human cathepsin L was determined, using a fluorogenic substrate.

Assay Buffer (pH 5.5):

20 mM sodium acetate (M=82.04; 164 mg), 4.0 mM EDTA (M=372.2; 149 mg), 0.012% Triton-X (390 μL 3%) dissolved in 100 mL H₂O and pH adjusted to 5.5.

Cathepsin L:

Human liver cathepsin L (Enzyme System Products, Catl-1; 5 μL of a 1.61 μU/μL stock) was solubilized in 2500 μL enzyme buffer. Prior to running experiment, 1 μL 0.5 M DTT/100 μL enzyme solution was added. Incubated for 5 minutes on ice, then added to reaction mixture.

Substrate:

Z-Phe-ARG-AFC*TFA (Enzyme System Products AF052; 15.6 mg) was dissolved in 1 mL DMSO, giving a 20 mM solution.

Assay Conditions:

The assay was performed in 96-well plates. 50 μL assay buffer was added to the well followed by 25 μL reference inhibitor (cystatin, stock 1 mg/mL, diluted with assay buffer to give 25 nM in assay) or a compound of the invention (diluted with assay buffer to give 10 μM in the assay). Then 25 μL (80 nU) enzyme was added, and 1 min. later (at 37° C.) 100 μL substrate (10 μM in the assay) was added. The excitation wavelength was 400 nm, and the emission was measured at 505 nm for 10-20 minutes at 37° C. Each measurement was made in duplicate. In the software (SOFTmax Pro) used for data collection from the fluorometer (Molecular Devices: Gemini XS), it was ensured that the measured slopes were linear (R²>0.99). Data were exported to GraphPad Prism and nonlinear regression was performed using the option Sigmoidal dose-response (variable slope).

Human CYP1A2 Assay

This assay was used to determine the effect of a compound of the invention on the CYP1A2 liver metabolising enzyme.

Vivid® CYP1A2 Blue Screening Kit from PanVera was used.

Kit Components:

Vivid® CYP450 Reaction Buffer I: 200 mM potassium phosphate buffer, pH 8. CYP1A2 baculosomes® reagent: CYP1A2 and NADH-P450 reductase (P450-specific content: 1.1 μM). Microsomes prepared from insect cells that were infected with baculovirus containing the cDNA for human CYP1A2 and rabbit cytochrome P450 reductase.

Regenerating System: 333 mM glucose-6-phosphate and 40 U/mL glucose-6-phosphate dehydrogenase in 100 mM potassium phosphate buffer, pH 8.

Vivid® CYP1A2 Blue Substrate.

Vivid® Blue Standard: 3-cyano-7-hydroxycoumarin.

10 mM NADP⁺ in 100 mM potassium phosphate buffer, pH 8.

Procedure:

Tubes thawed on ice. 2× reaction buffer dispensed (room temperature) in two eppendorf tubes (for Mix A and Mix B).

Controls or compounds of the invention: Diluted to a concentration of 50 μM. Positive control compound: α-naphthoflavone.

Negative controls: DMSO and H₂O.

Mix A: 485 μL reaction buffer, 10 μL regeneration system, 5 μL Baculosome reagent.

Mixed gently and placed on ice.

Mix B: 88.5 μL reaction buffer, 1.5 μL substrate, 10 μL 10 mM NADP⁺.

In a 96-well plate (Costar c-3904), 40 μL of 50 μM sample dispensed. 50 μl Mix A added. Incubated 20 min. at rt. Then, 10 μl of Mix B added. Fluorescence determined (Molecular Devices, SPECTRAmax Gemini EM): Ex: 409 nm, Em: 460 nm, at 37° C. for 20 min. (Automix: 5 s, top read).

Following the procedure for CYP1A2, using the appropriate kits below, the effect of a compound of the invention on the other CYP enzymes was determined:

Vivid® CYP2C9 Red Screening Kit from PanVera.

Vivid® CYP2C19 Red Screening Kit from PanVera.

Vivid® CYP2D6 Cyan Screening Kit from PanVera.

Vivid® CYP3A4 Red Screening Kit from PanVera.

Results:

| Assay | Enzyme/species | Example 5 | |
|---|---|---|---|
| Primary enzymatic | DPP-I | 13 ± 3 | nM |
| screening (IC₅₀) | | | |
| Secondary enzymatic | Cathepsin B | >10 | μM |
| screening | Cathepsin H | >10 | μM |
| (10 μM cutoff or IC₅₀) | Cathepsin L | >10 | μM |
| Metabolism: CYP enzymes | CYP1A2 | 14896 ± 556 | nM |
| (20 μM cutoff or IC₅₀) | CYP2C9 | >20 | μM |
| | CYP2C19 | >20 | μM |
| | CYP2D6 | 12605 ± 677 | nM |
| | CYP3A4 | 3604 ± 3410 | nM |

The results show that the compound tested is a selective inhibitor of DPP-I.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

1. A compound of formula (I) 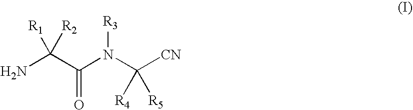
or a pharmaceutically acceptable salt or prodrug thereof, wherein
R1 is hydrogen, C1-6alkyl optionally substituted with a substituent selected from the group consisting of halogen, amino, hydroxy, cyano and C1-3alkoxy; or C2-6alkenyl, C2-6alkynyl, C1-6alkoxy, C1-6alkylthio, C1-6alkylcarbonyl, an unsubstituted or substituted C3-10cycloalkyl group, an unsubstituted or substituted C3-10cycloalkylcarbonyl group, an unsubstituted or substituted C5-10cycloalkenyl group, an unsubstituted or substituted C3-7heterocycloalkyl group, an unsubstituted or substituted C1-6alkylaryl group, an unsubstituted or substituted C2-6alkenylaryl group, an unsubstituted or substituted C1-6alkylheteroaryl group, an unsubstituted or substituted aryl group, an unsubstituted or substituted heteroaryl group, an unsubstituted or substituted aroyl group, an unsubstituted or substituted arylthio group, an unsubstituted or substituted aryloxy group, an unsubstituted or substituted arylsulfonyl group, an unsubstituted or substituted arylamino group, an unsubstituted or substituted heteroaroyl group, an unsubstituted or substituted heteroaryloxy group, an unsubstituted or substituted heteroarylsulfonyl group, an unsubstituted or substituted heteroarylamino group, an unsubstituted or substituted C1-5alkylC3-7cycloalkyl group or an unsubstituted or substituted C1-5alkylC3-7heterocycloalkyl group;
R2 is hydrogen or C1-6alkyl; or R1 and R2 together form an unsubstituted or substituted C3-10cycloalkyl group or an unsubstituted or substituted C3-7heterocycloalkyl group;
R3 is hydrogen or C1-6alkyl; or R1 and R3 together form an unsubstituted or substituted C3-7heterocycloalkyl group;
R4 is hydrogen, C1-6alkyl, C2-6alkenyl, C2-6alkynyl, C1-6alkoxy, C1-6alkylthio, C1-6alkylcarbonyl, C1-6alkylsulfonyl, an unsubstituted or substituted C3-10cycloalkyl group, an unsubstituted or substituted C3-10cycloalkylcarbonyl group, an unsubstituted or substituted C5-10cycloalkenyl group, an unsubstituted or substituted C3-7heterocycloalkyl group, an unsubstituted or substituted C1-6alkylaryl group, an unsubstituted or substituted C2-6alkenylaryl group, an unsubstituted or substituted C1-6alkylheteroaryl group, an unsubstituted or substituted aryl group, an unsubstituted or substituted heteroaryl group, an unsubstituted or substituted aroyl group, an unsubstituted or substituted arylthio group, an unsubstituted or substituted aryloxy group, an unsubstituted or substituted arylsulfonyl group, an unsubstituted or substituted arylamino group, an unsubstituted or substituted heteroaroyl group, an unsubstituted or substituted heteroaryloxy group, an unsubstituted or substituted heteroarylsulfonyl group, an unsubstituted or substituted heteroarylamino group, an unsubstituted or substituted C1-5alkylC3-7cycloalkyl group or an unsubstituted, substituted C1-5alkylC3-7heterocycloalkyl group or a group of the formula: 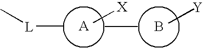
wherein A is a ring system with one ore more substituents X, and A is selected from C5-7cycloalkyl, C5-7heterocycloalkyl, aryl and heteroaryl;
X being the same or different selected from hydrogen, Cl, Br, F, I, hydroxy, amino, cyano, trifluoromethyl, C1-6alkyl, C1-6alkylthio or C1-6alkoxy;
B is a ring system with one ore more substituents Y, and B is selected from C5-7cycloalkyl, C5-7heterocycloalkyl, aryl and heteroaryl;
Y being the same or different selected from hydrogen, Cl, Br, F, I, hydroxy, amino, cyano, trifluoromethyl, C1-6alkyl, C1-6alkylthio or C1-6alkoxy;
-L- is a linker, which is C1-6alkyl or C2-6alkenyl, or a moiety selected from the group consisting of 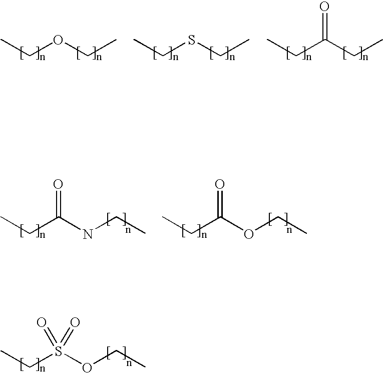
and, wherein the linker -L- may be attached via either of the two free bonds to the ring A;
n is the same or different integer selected from 0, 1, 2 and 3;
R5 is hydrogen or C1-6alkyl; or R4 and R5 together form an unsubstituted or substituted C3-10cycloalkyl group or an unsubstituted or substituted C₃₋₇heterocycloalkyl group;
wherein a substituted group is substituted with one, two or three substituents independently selected from the group consisting of C1-6alkyl, C1-6alkoxy, C1-6alkylthio, C1-6alkylcarbonyl, C1-6-N-alkylamide, dialkylamino-C1-6alkyl, amide, hydroxy, carboxy, amino, nitro, halogen, trifluoromethyl, trifluoromethoxy, trifluoromethylthio and cyano.

2. A compound according to claim 1, wherein R1 is selected from the group consisting of hydrogen, C1-6alkyl, an unsubstituted or substituted aryl, an unsubstituted or substituted C1-6alkylaryl group, an unsubstituted or substituted C1-6alkylheteroaryl group, or an unsubstituted or substituted C3-10-cycloalkyl group.

3. A compound according to claim 1 or 2, wherein R1 is hydrogen, methyl, ethyl, n-propyl, isopropyl, n-butyl, isobutyl, sec-butyl, tert-butyl, phenyl, benzyl or cyclohexyl.

4. A compound according to claim 1, wherein R1 is hydrogen, methyl or ethyl.

5. A compound according to claim 1, wherein R1 and R2 together form an unsubstituted or substituted C3-10cycloalkyl group or an unsubstituted or substituted C3-7heterocycloalkyl group.

6. A compound according to claim 1, wherein R1 and R2 together form an unsubstituted or substituted cyclohexyl group.

7. A compound according to claim 1, wherein R1 and R3 together form an unsubstituted or substituted C3-7heterocycloalkyl group.

8. A compound according to claim 1, wherein R1 and R3 together form a pyrrolidonyl or piperidonyl.

9. A compound according to claim 1, wherein R2 is hydrogen.

10. A compound according to claim 1, wherein R3 is hydrogen or methyl.

11. A compound according to claim 1, wherein R4 is selected from the group consisting of hydrogen, C1-6alkyl, an unsubstituted or substituted C1-6alkylaryl group, an unsubstituted or substituted C1-6alkenylaryl group and an unsubstituted or substituted C1-6alkylheteroaryl group.

12. A compound according to claim 1, wherein R4 is hydrogen, unsubstituted or substituted benzyl, 2-phenylethyl, 3-phenylprop-2-ene, [1,1′-biphenyl-4-yl]methyl or [1,1′-biphenyl-2-yl]methyl.

13. A compound according to claim 1, wherein R5 is hydrogen.

14. A compound according claim 1, wherein R4 and R5 together form an unsubstituted or substituted C3-10cycloalkyl group or an unsubstituted or substituted C3-7heterocycloalkyl group.

15. A compound according to claim 1, wherein at least one of R4 and R5 is hydrogen.

16. A compound according to claim 1 with the following structure 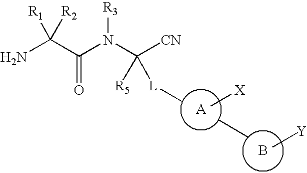
wherein R1, R2, R₃, R₅, A, B, X, Y and L are defined in claim 1.

17. A compound according to claim 1, wherein R4 is
[1,1′-biphenyl-4-yl]methyl, [1,1′,2-methylbiphenyl-4-yl]methyl, [1,1′,3-methylbiphenyl-4-yl]methyl, [1,1′,2-hydroxybiphenyl-4-yl]methyl, [1,1′,3-hydroxybiphenyl-4-yl]methyl, [1,1′,2-methoxybiphenyl-4-yl]methyl, [1,1′,3-methoxybiphenyl-4-yl]methyl, [1,1′,2-methylthiobiphenyl-4-yl]methyl, [1,1′,3-methylthiobiphenyl-4-yl]methyl, [1,1′,2-cyanobiphenyl-4-yl]methyl, [1,1′,3-cyanobiphenyl-4-yl]methyl, [1,1′,2-aminobiphenyl-4-yl]methyl, [1,1′,3-aminobiphenyl-4-yl]methyl, [1,1′,2-fluorobiphenyl-4-yl]methyl, [1,1′,3-fluorobiphenyl-4-yl]methyl, [1,1′,2-chlorobiphenyl-4-yl]methyl, [1,1′,3-chlorobiphenyl-4-yl]methyl, [1,1′,2-bromobiphenyl-4-yl]methyl, [1,1′,3-bromobiphenyl-4-yl]methyl, [1,1′,2′-fluorobiphenyl-4-yl]methyl, [1,1′,3′-fluorobiphenyl-4-yl]methyl, [1,1′,4′-fluorobiphenyl-4-yl]methyl, [1,1′,2′-chlorobiphenyl-4-yl]methyl, [1,1′,3′-chlorobiphenyl-4-yl]methyl, [1,1′,4′-chlorobiphenyl-4-yl]methyl, [1,1′,2′-bromobiphenyl-4-yl]methyl, [1,1′,3′-bromobiphenyl-4-yl]methyl, [1,1′,4′-bromobiphenyl-4-yl]methyl, [1,1′,2′-cyanobiphenyl-4-yl]methyl, [1,1′,3′-cyanobiphenyl-4-yl]methyl, [1,1′,4′-cyanobiphenyl-4-yl]methyl, [1,1′,4′-hydroxybiphenyl-4-yl]methyl, [1,1′,4′-aminobiphenyl-4-yl]methyl, [1,1′,4′-methoxybiphenyl-4-yl]methyl, [1,1′,4′-methylthiobiphenyl-4-yl]methyl, [1,1′,4′-trifluoromethylbiphenyl-4-yl]methyl, [1,1′,2-methyl-4′-fluorobiphenyl-4-yl]methyl, [1,1′,2-chloro-4′-cyanobiphenyl-4-yl]methyl, [1,1′,2-methoxy-3′-fluorobiphenyl-4-yl]methyl, [1,1′,2-hydroxy-2′-fluorobiphenyl-4-yl]methyl, [1,1′,3-amino-3′-methoxybiphenyl-4-yl]methyl, [1,1′,2-fluoro-4′-fluorobiphenyl-4-yl]methyl [2-phenyl-1,3-thiazol-4-yl]methyl, [5-phenylpyridin-3-yl]methyl, [3-pyrimidin-5-ylphenyl]methyl, [3-pyridin-2-ylphenyl]methyl, [3-pyridin-4-ylphenyl]methyl, [3-(1H-indol-6-yl)phenyl]methyl, [1-(2-fluorophenyl)piperidin-4-yl]methyl, [3-fluoro-4-(1-piperidinyl)phenyl]methyl, [1,1′-biphenyl-4-yl]ethyl, [1,1′,2-methylbiphenyl-4-yl]ethyl, [1,1′,3-methylbiphenyl-4-yl]ethyl, [1,1′,2-hydroxybiphenyl-4-yl]ethyl, [1,1′,3-hydroxybiphenyl-4-yl]ethyl, [1,1′,2-methoxybiphenyl-4-yl]ethyl, [1,1′,3-methoxybiphenyl-4-yl]ethyl, [1,1′,2-methylthiobiphenyl-4-yl]ethyl, [1,1′,3-methylthiobiphenyl-4-yl]ethyl, [1,1′,2-cyanobiphenyl-4-yl]ethyl, [1,1′,3-cyanobiphenyl-4-yl]ethyl, [1,1′,2-aminobiphenyl-4-yl]ethyl, [1,1′,3-aminobiphenyl-4-yl]ethyl, [1,1′,2-fluorobiphenyl-4-yl]ethyl, [1,1′,3-fluorobiphenyl-4-yl]ethyl, [1,1′,2-chlorobiphenyl-4-yl]ethyl, [1,1′,3-chlorobiphenyl-4-yl]ethyl, [1,1′,2-bromobiphenyl-4-yl]ethyl, [1,1′,3-bromobiphenyl-4-yl]ethyl, [1,1′,2′-fluorobiphenyl-4-yl]ethyl, [1,1′,3′-fluorobiphenyl-4-yl]ethyl, [1,1′,4′-fluorobiphenyl-4-yl]ethyl, [1,1′,2′-chlorobiphenyl-4-yl]ethyl, [1,1′,3′-chlorobiphenyl-4-yl]ethyl, [1,1′,4′-chlorobiphenyl-4-yl]ethyl, [1,1′,2′-bromobiphenyl-4-yl]ethyl, [1,1′,3′-bromobiphenyl-4-yl]ethyl, [1,1′,4′-bromobiphenyl-4-yl]ethyl, [1,1′,2′-cyanobiphenyl-4-yl]ethyl, [1,1′,3′-cyanobiphenyl-4-yl]ethyl, [1,1′,4′-cyanobiphenyl-4-yl]ethyl, [1,1′,4′-trifluoromethylbiphenyl-4-yl]ethyl, [1,1′,2-methyl-4′-fluorobiphenyl-4-yl]ethyl, [1,1′,2-chloro-4′-cyanobiphenyl-4-yl]ethyl, [1,1′,2-methoxy-3′-fluorobiphenyl-4-yl]ethyl, [1,1′,2-hydroxy-2′-fluorobiphenyl-4-yl]ethyl, [1,1′,3-amino-3′-methoxybiphenyl-4-yl]ethyl, [2-phenyl-1,3-thiazol-4-yl]ethyl, [5-phenylpyridin-3-yl]ethyl, [3-pyrimidin-5-ylphenyl]ethyl, [3-pyridin-2-ylphenyl]ethyl, [3-pyridin-4-ylphenyl]ethyl, [3-(1H-indol-6-yl)phenyl]ethyl, [1-(2-fluorophenyl)piperidin-4-yl]ethyl, [3-fluoro-4-(1-piperidinyl)phenyl]ethyl, [1,1′-biphenyl-4-yl]methyloxymethyl, [1,1′,4′-fluorobiphenyl-4-yl]methyloxymethyl, [1,1′-biphenyl-4-yl]methylthiomethyl, [1,1′,4′-fluorobiphenyl-4-yl]methylthiomethyl, [1,1′-biphenyl-4-yl]ethylenyl or [1,1′,4′-fluorobiphenyl-4-yl]ethylenyl.

18. A compound according to claim 1, selected from the group consisting of
N-(2S-2-amino-3-phenylpropionyl)-aminoacetonitrile;
(2S)—N-[(2S)-2-aminobutanoyl]-2-amino-3-phenylpropionitrile;
(2S)—N-Methyl-N-[(2S)-2-aminobutanoyl]-2-amino-3-phenylpropionitrile;
(2S)—N-[(2S)-2-aminobutanoyl]-2-amino-3-(p-chlorophenyl)propionitrile;
(2S)—N-[(2S)-2-aminobutanoyl]-2-amino-3-(1,1′-biphenyl-4-yl)propionitrile;
(2S)-(4Z)-N-[(2S)-2-aminobutanoyl]-2-amino-5-phenyl-pent-4-ene-nitrile;
(2S)—N-[(2S)-2-aminobutanoyl]-2-amino-4-phenylbutyronitrile and
(2S)—N-[(2S)-3-phenylaminopropanoyl]-2-amino-3-phenylpropionitrile.

19. The compound according to claim 1, which exhibits an IC50 value of 500 μM or less such as, e.g., 100 μM or less, 50 μM or less, 1 μM or less, 500 nM or less, 100 nM or less, 75 nM or less, 50 nM or less, or 25 nM or less.

20. A compound according to claim 1 for use in medicine.

21. A compound according to claim 20 for use as a protease inhibitor.

22. A compound according to claim 21 for use as a cysteine protease inhibitor.

23. A compound according to claim 20 for use in the treatment, prophylaxis and/or diagnosis of inflammation, type2 diabetes, asthma, severe influenza, respiratory syncytial virus infection, CD8 T cell inhibition, inflammatory bowel diseases, psoriasis, atopic dermatitis, Papillon Lefevre syndrome, Haim Munk syndrome, gum disease, periodontitis, rheumatoid arthritis, Huntington's disease, Chagas' disease, Alzheimer's disease, sepsis or for application in target cell apoptosis.

24. A pharmaceutical composition comprising, as an active substance, a compound as defined in claim 1 or a pharmaceutically acceptable salt thereof together with a pharmaceutically acceptable carrier or diluent.

25. A pharmaceutical composition according to claim 24 in unit dosage form, comprising from about 1 mg to about 1000 mg such as, e.g., from about 10 mg to about 500 mg, from about 0.05 to about 100 mg or from about 0.1 to about 50 mg, of the active substance.

26. A pharmaceutical composition according to claim 24 or 5 for oral, nasal, transdermal, pulmonal or parenteral administration.

27. A method for the treatment of ailments, the method comprising administering to a subject in need thereof an effective amount of a compound as defined in claim 1 or of a composition.

28. The method according to claim 27, wherein the effective amount of the compound is in a range of from about 1 mg to about 1000 mg such as, e.g., from about 10 mg to about 500 mg, from about 0.05 to about 100 mg or from about 0.1 to about 50 mg per day.

29. Use of a compound as defined in claim 1 for the preparation of a medicament.

30. Use of a compound as defined in claim 1 for the preparation of a medicament for treatment, prophylaxis and/or diagnosis of inflammation, type2 diabetes, asthma, severe influenza, respiratory syncytial virus infection, CD8 T cell inhibition, inflammatory bowel diseases, psoriasis, atopic dermatitis, Papillon Lefevre syndrome, Haim Munk syndrome, gum disease, periodontitis, rheumatoid arthritis, Huntington's disease, Chagas' disease, Alzheimer's disease, sepsis or for application in target cell apoptosis.

31. A method for modulating DPP-I levels in a subject in need thereof comprising administering to said subject an amount of a compound as defined in claim 1 or a composition in an amount effective to modulate said DPP-I levels in said subject.

32. A method according to claim 31, wherein said DPP-I is inhibited.

33. A method according to claim 32, wherein DPP-I is selectively inhibited as determined by IC50(Cathepsin B)/IC50(DPP-I assay) is 25 or more such as, e.g., 50 or more, 75 or more, 100 or more, 250 or more, 500 or more or 750 or more.

34. The method according to claim 32, wherein DPP-I is selectively inhibited as determined by IC50(Cathepisn H)/IC50(DPP-I assay) is 25 or more such as, e.g., 50 or more, 75 or more, 100 or more, 250 or more, 500 or more or 750 or more.

35. The method according to claim 32, wherein DPP-I is selectively inhibited as determined by IC50(Cathepsin L)/IC50(DPP-I assay) is 25 or more such as, e.g., 50 or more, 75 or more, 100 or more, 250 or more, 500 or more or 750 or more.
